(19) United States
(12) Patent Application Publication
(10) Pub. No.: US 2004/0110173 A1
(43) Pub. Date: Jun. 10, 2004

(54) Haplotype partitioning in the proximal promoter of the human growth hormone (GH1) gene

(76) Inventors: David Neil Cooper, Cardiff (GB); Ann Marie Procter, Cardiff (GB); David Stuart Millar, Cardiff (GB); John Gregory, Cardiff (GB)

Correspondence Address: QUARLES & BRADY LLP, 411 E. WISCONSIN AVENUE, SUITE 2040, MILWAUKEE, WI, 53202-4497, US

(21) Appl. No.: 10/459,184

(22) Filed: Jun. 11, 2003

(30) Foreign Application Priority Data
Dec. 9, 2002 (GB) 0229725.7
Mar. 20, 2003 (GB) 0306417.7
Apr. 10, 2003 (GB) 0308240.1

(51) Int. Cl. C12Q 1/68

(52) U.S. Cl. 435/6

(57) ABSTRACT

The invention relates to variants of the human growth gene (GH1) and, in particular, variants in the proximal promoter region thereof. Moreover, the invention relates to the interaction of said variants and how said interaction affects growth hormone expression.

### CROSS-REFERENCE TO RELATED APPLICATION

This application claims priority to British Patent Application 0229725.7, filed 19 Dec. 2002; British Patent Application 0306417.7, filed 20 Mar. 2003; and British Patent Application 0308240.1, filed 10 Apr. 2003.

### STATEMENT REGARDING FEDERALLY SPONSORED RESEARCH OR DEVELOPMENT

### BACKGROUND OF THE INVENTION

The invention concerns a method for diagnosing the existence of, or a susceptibility to, growth hormone dysfunction and a kit, including the parts thereof, suitable for use therein and further research tools based thereon.

Human stature is a highly complex trait resulting from the interaction of multiple genetic and environmental factors. Since familial short stature is already known to be associated with inherited mutations of the growth hormone (GH1) gene, it appears reasonable to suppose that polymorphic variation in this pituitary-expressed gene can also influence adult height.

The human GH1 gene is located on chromosome 17q23 within a 66 kb cluster of five related genes including the placentally expressed growth hormone gene (GH2; MIM #139240), two chorionic somatomammotropin genes (CSH1 and CSH2) and a pseudogene (CSHP1). The proximal region of the GH1 gene promoter exhibits a high level of sequence variation with 16 single nucleotide polymorphisms (SNPs) having been reported within a 535 base-pair stretch. The majority of these SNPs occur at the same positions in which the GH1 gene differs from the paralogous GH2, CSH1, CSH2 and CSHP1 genes, suggesting that they may have arisen through gene conversion.

The expression of the human GH1 gene is also influenced by a Locus Control Region (LCR) located between 14.5 kb and 32 kb upstream of the GH1 gene. The LCR contains multiple DNase I hypersensitive sites and is required for the activation of the genes of the GH gene cluster in both pituitary and placenta. Two DNase I hypersensitive sites (I and II) contain binding sites for the pituitary-specific transcription factor Pit-1 and are responsible for the high level-, somatotrope-specific expression of the GH1 gene.

Somewhat unusually, we have undertaken investigations to assess the functional importance of the polymorphic variation in both the proximal promoter region and the LCR of the GH1 gene.

As a result of the investigations described herein, we have shown in our study population that variation occurred at 15 of the 16 known SNP locations and manifested itself in a total of 40 different promoter haplotypes. Further, investigation of these haplotypes enabled us to partition them and so conclude that 6 of the SNP's act as major determinants of GH1 gene expression, whilst a further 6 SNP's are only marginally informative of GH1 gene expression.

Moreover, given the genetic complexity of human stature, our data have led us to conclude that certain combinations of SNP's, and so haplotypes, can have significantly determinative effects on human stature. Accordingly, knowledge of this information is useful for identifying individuals who suffer from under-expression of growth hormone and so require replacement therapy at least until puberty.

In the field of medical genetics, where an individuals' DNA is assayed in order to determine whether there are any lesions that affect the structure, function or expression of the growth hormone (GH1) gene, it is relatively straightforward to detect any of the gross deletions or major mutations. However, as our data show, an individual may under-express growth hormone because of the nature of the GH1 promoter haplotype. Using conventional genetic assays, such an individual, if not possessing any of the major deletions or mutations, would be considered to be normal for growth hormone expression. However, the work described herein has elucidated the combination of SNP's that affect growth hormone expression and, in turn, stature. This knowledge can be used to generate a GH assay that is sensitive to GH1 expression of the wild-type and mutated gene and so accurate for use in the genetic testing of a wide range of individuals including those that do not manifest the symptoms associated with the gross gene deletions.

### SUMMARY OF THE INVENTION

In one embodiment, the present invention is a method for diagnosing the existence of, or a susceptibility to, growth hormone dysfunction in an individual comprising: (a) obtaining a test sample of a nucleic acid molecule encoding the proximal promoter region of the growth hormone gene (GH1) from an individual to be tested; (b) examining said nucleic acid molecule for a plurality of the following six SNP's: 1, 6, 7, 9, 11 and 14 (described in Table 1), or the corresponding haplotypes thereof (also described in Table 1); or a polymorphism in linkage disequilibrium therewith; (c) and where a plurality of said SNP's, or their said corresponding haplotypes, or their said corresponding polymorphisms, exist determining that the individual may be suffering from, or has a susceptibility to, growth hormone dysfunction. Preferably, said polymorphism is at 114 of the locus control region of the said gene or at 1194 of the locus control region of said gene.

In another embodiment, the present invention is a method for diagnosing the existence of, or a susceptibility to, growth hormone dysfunction in an individual, comprising: (a) obtaining a test sample of a nucleic acid molecule encoding the proximal promoter region of the growth hormone gene (GH1) from an individual to be tested; (b) examining said nucleic acid molecule for any one or more of the following haplotypes in Table 1 indicated as numbers 3, 4, 5, 7, 11, 13, 17, 19, 23, 24, 26 or 29; (c) and where said haplotypes exist determine that the individual may be suffering from, or has a susceptibility to, growth hormone dysfunction.

Preferably the “examining” step above involves amplification of the gene and the amplification primer is selected from the group consisting of: GGG AGC CCC AGC AAT GC (GH1F); and TGT AGG MG TCT GGG GTG C (GH1R).

In another embodiment, the present invention is a kit suitable for carrying out diagnostic methods described above. In one embodiment the kit comprises: (a) at least one of the following primers for detecting and/or amplifying the proximal promoter region of the growth hormone gene (GH1); GGG AGC CCC AGC MT GC (GH1F); TGT AGG MG TCT GGG GTG C (GH1R); and, optionally, (b) one or more reagents suitable for carrying out PCR for amplifying desired regions of the patient's DNA. Additionally or alternatively, other primers could be used that are complementary to selected regions of the gene containing the SNP's defined herein as 1, 6, 7, 9, 11 and 14.

In another embodiment, the present invention is a vector comprising at least the proximal promoter region of GH1 wherein said region comprises a plurality of the following SNP's: 1, 6, 7, 9, 11 and 14. Preferably, said region comprises SNP's 6 and 9, SNP's 10 and 12, or SNP's 8 and 11. In another embodiment, said region is characterised by any one or more of the following haplotypes shown in Table 1: 3, 4, 5, 7, 11, 13, 17, 19, 23, 24, 26 or 29. In one embodiment, the vector further comprises a GH1 locus control region proximal promoter fusion construct.

In one embodiment, said proximal promoter region of the vector is functionally linked to the coding region of a selected gene wherein the activity of the said proximal promoter can be monitored. In one embodiment, said proximal promoter region is linked to the coding region of the growth hormone gene (GH1). In another embodiment, said proximal promoter region in said gene is further linked to a tag, preferably a protein tag, whereby the expression of said gene, and so the activity of said proximal promoter region, can be monitored.

In another embodiment, the vector is further provided with at least one further proximal promoter region of the growth hormone gene (GH1), preferably where said additional proximal promoter region differs from that of the original proximal promoter region. Preferably, each proximal promoter region is linked to a different coding sequence and/or linked, either directly or indirectly, to a different tag that is capable of monitoring the activities of each of the said promoters.

In another embodiment, the present invention is a host cell transformed with any of the vectors described above.

In another embodiment, the present invention is a recombinant cell line that is engineered to express a reporter molecule whose expression is under the control of the proximal promoter of the growth hormone gene wherein said proximal promoter comprises a plurality of the following SNP's: 1, 6, 7, 9, 11 or 14 and/or any one or more of the following haplotypes: 3, 4, 5, 7, 11, 13, 17, 19, 23, 24, 26 or 29 shown in Table 1.

In another embodiment, the present invention is a transgenic non-human animal which under expresses growth hormone as a result of having a GH1 promoter containing a plurality of the following SNP's: 1, 6, 7, 9, 11 and 14 and/or as a result of said promoter being characterised by one of the following haplotypes: 3, 4, 5, 7, 11, 13, 17, 19, 23, 24, 26 or 29, shown in Table 1. Preferably, said promoter is characterised by haplotype 23, 27 or 1.

In another embodiment, the present invention is an artificial proximal promoter region of the growth hormone gene (GH1) characterised by the haplotype AGGGGTTAT-ATGGAG, the haplotype AG-TTGTGGGACCACT, or the haplotype AG-TTTTGGGGCCACT.

In another embodiment, the present invention is a method for screening therapeutically active drugs which can be used to treat growth hormone dysfunction comprising exposing a cell or cell line described above to a candidate drug and then determining if the candidate drug has affected the activity of the promoter region of the growth hormone gene and so, in the case of the cell line, the expression of the reporter molecule.

In another embodiment, the present invention is a method for screening for therapeutically active drugs which can be used to treat growth hormone dysfunction comprising exposing a transgenic non-human animal described above to candidate drugs and then monitoring the growth of said animal and where the candidate drug is shown to have a positive effect, in terms of animal growth, concluding that said growth is indicative of the therapeutic activity of said candidate drug.

Other objects, functions and embodiments of the present invention will become apparent to one of skill in the art after examination of the specification, claims and drawings.

### BRIEF DESCRIPTION OF THE SEVERAL VIEWS OF THE DRAWINGS

FIG. 1 illustrates the location of 16 SNPs in the GH1 promoter relative to the transcriptional start site (denoted by an arrow). The hatched box represents exon 1. The positions of the binding sites for transcription factors, nuclear factor 1 (NF1), Pit-1, and vitamin D receptor (VDRE), the TATA baox, and the translational initiation codon (ATG) are also shown.

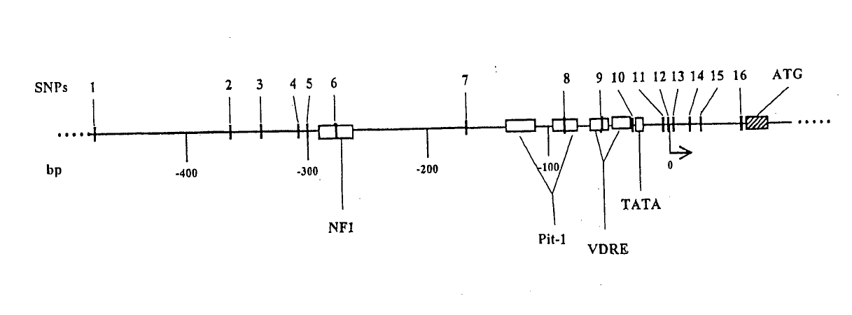

FIG. 2 illustrates normalized expression levels of the 40 GH1 haplotypes relative to the wild-type (haplotype 1). Haplotypes associates with a significantly reduced level of luciferase reporter gene expression (by comparison with haplotype 1) are denoted by hatched bars. Haplotypes associated with a significantly increased level of luciferase reporter gene expression (by comparison with haplotype 1) are denoted by solid bars. Haplotypes are arranged in decreasing order of prevalence.

FIG. 3 illustrates minimum relative residual deviance _{δR}(π_{k,min}) of normalized expression levels associated with haplotye partitioning using k SNPs (shaded bars). The dotted curve depicts the number of haplotypes comprising the minimum-_{δR} partitioning π_{k,min}.

FIG. 4 illustrates regression tree of GH1 gene promoter expression as obtained by recursive binary haplotype partitioning, using six selected SNPs (nos. 1, 6, 7, 9, 11, and 14). Numbers on nodes refer to the SNPs by which the respective nodes were split. Terminal nodes (“leafs”) are depicted as squares and numbered from left to right.

FIG. 5 illustrates “Reduced Median Network” connecting the seven haplotypes (circles) that have been observed at least 8 times in 154 male Caucasians. The size of each circle is proportional to the frequency of the respective haplotype in the control sample. Haplotypes H12 and H23 have been included as connecting nodes even although hey have been observed only 5 and 2 times, respectively. SNPs at which haplotypes differ are given alongside each branch. The dark dot marks a non-observed haplotype or a double mutation at SNP sites 4 and 5.

FIG. 6 illustrates differences in protein binding capacity between GH1 promoter SNP alleles revealed by electrophoretic mobility shift (EMSA) assays. Arrows denote allele-specific interacting proteins. The arrowhead denotes the position of a Pit-1-like binding protein. −ve (negative control), +ve (positive control), S (specific competitor), N (non-specific competitor), P (Pit-1 consensus sequence), P* (prolactin gene Pit-1 binding site), TSS (transcriptional initiation site).

### DESCRIPTION OF THE INVENTION

Accordingly, the present invention concerns a method for diagnosing the existence of, or a susceptibility to, growth hormone dysfunction in an individual comprising:

a) obtaining a test sample of a nucleic acid molecule encoding the proximal promoter region of the growth hormone gene (GH1) from an individual to be tested;

b) examining said nucleic acid molecule for a plurality of the following 6 SNP's: 1, 6, 7, 9, 11 and 14 (described in Table 1), or the corresponding haplotypes thereof (also described in Table 1); or a polymorphism in linkage disequilibrium therewith;

c) and where a plurality of said SNP's, or their said corresponding haplotypes, or their said corresponding polymorphisms, exist determining that the individual may be suffering from, or has a susceptibility to, growth hormone dysfunction.

By “plurality” we mean that at least two SNPs, etc., will be present. In a preferred method of the invention said polymorphism in linkage disequilibrium is the polymorphism at 1144 or 1194 of the corresponding locus contro region, as herein described.

According to a further aspect, or embodiment, of the invention there is provided a method for diagnosing the existence of, or a susceptibility to, growth hormone dysfunction in an individual comprising:

a) obtaining a test sample of a nucleic acid molecule encoding the proximal promoter region of the growth hormone gene (GH1) from an individual to be tested;

b) examining said nucleic acid molecule for any one or more of the haplotypes in Table 1 indicated as Nos. 3, 4, 5, 7, 11, 13, 17, 19, 23, 24, 26 or 29;

c) and where said haplotype exists determining that the individual may be suffering from, or has a susceptibility to, growth hormone dysfunction.

Our investigations have led us to conclude that these haplotypes are responsible for a reduction in growth hormone expression and therefore lead to growth hormone dysfunction

Preferably, conventional means are used for performing the diagnostic method of the invention and so, typically, examining said nucleic acid molecule of an individual to be tested will involve the amplification of same using primers, or pairs of primers, which hybridise to the complementary strand of nucleic acid to be amplified. Examples of suitable primers are given below:

GGG AGC CCC AGC AAT GC (GH1F);
and/or
TGT AGG AAG TCT GGG GTG C (GH1R).

Advantageously, the primers are labelled, in order to enable their detection, using conventional labels such as radio labels, enzymes, fluorescent or chemiluminescent labels or biotin-avidin labels.

Most suitably the primers hybridise to the nucleic acid molecule under stringent conditions. This means that the level of hybridisation is sufficient to distinguish between the 5 homologous genes within the 66 kb cluster on chromosome 17q23. Generally, the washing conditions that support stringent hybridisation should be a combination of temperature and salt concentration so that the denaturation temperature is approximately 5 to 20° C. below the calculated melt temperature of the nucleic acid under study.

According to a further aspect of the invention there is provided a kit suitable for carrying out the aforementioned diagnostic methods of the invention which kit comprises:

a) at least one of the following primers for detecting and/or amplifying the proximal promoter region of GH1;

GGG AGC CCC AGC AAT GC (GH1F);
TGT AGG AAG TCT GGG GTG C (GH1R);

and, optionally,

b) one or more reagents suitable for carrying out PCR for amplifying desired regions of the patient's DNA.

Advantageously, the kit of the invention comprises oligonucleotides that are complementary to a plurality of the following SNP's: 1, 6, 7, 9, 11 and 14.

The SNP's and haplotypes of the invention have utility in the identification of therapies for the treatment of growth hormone dysfunction. It therefore follows that the insertion of one or more growth hormone genes, or parts thereof, comprising the aforementioned SNP's, and/or haplotypes, into suitable cells or cell lines will produce useful tools for identifying agents for treating growth hormone dysfunction. Therefore, according to a further aspect of the invention there is provided vector comprising at least the proximal promoter region of GH1 wherein said region comprises a plurality of the following SNP's: 1, 6, 7, 9, 11 and 14.

In a preferred embodiment of the invention said region comprises a plurality of the aforementioned SNP's and most ideally still 6 and 9; and/or 10 and 12; and/or 8 and 11. There is not only interaction (partitioning) within one promoter haplotype on one allele but also between promoter haplotypes, viz the promoter haplotype on the other allele. Moreover, there is some degree of parentally derived dominance, the paternal derived haplotype being more dominant than the maternal, or vice versa.

According to a further aspect of the invention there is provided a vector comprising at least a proximal promoter region of GH1 wherein said region is characterised by possessing any one or more of the following haplotypes shown in Table 1: 3, 4, 5, 7, 11, 13, 17, 19, 23, 24, 26 or 29.

According to a yet further aspect of the invention there is provided a vector comprising an LCR proximal promoter fusion construct as herein described.

Most preferably the vector is adapted for transforming or transfecting a prokaryotic or eukaryotic cell and is further provided with means for ensuring the activity of the promoter region can be monitored in response to agents that activate or inhibit same. Accordingly, said proximal promoter region is linked to the coding region of the growth hormone (GH1) gene or the coding region of an alternative gene whereby the expression of the growth hormone gene or the alternative gene can be used to monitor the activity of the corresponding promoter.

More ideally still, within the vector, the gene may be expressed upstream or downstream of an expression protein tag, for example, such a tag would be green fluorescent protein whereby expression of said GH1 coding region and its neighbouring tag is under the control of the proximal promoter of GH1.

In a further aspect or embodiment of the invention there is provided a vector comprising a plurality of promoters of the growth hormone gene (GH1) and most ideally a plurality of different promoters of the growth hormone gene. By the term different we mean each promoter will have a different coding sequence and thus comprise different types of SNP's, and so haplotypes. In this arrangement, most advantageously, each promoter is either linked to a different DNA sequence whereby the promoter activity can be monitored as a result of the expression of different genes, or alternatively, the same coding sequence may be used but it is suitably provided with a different tag whereby the expression of the same gene can be differentially monitored using the different tags.

These vectors of the invention are ideally used to transform host cells which can, advantageously, be used for the purpose of screening agents that may be useful in treating growth hormone dysfunction. The preferred cells include bacterial yeast, fungus, insect cells, or mammalian cells, and most preferably immortalised cells such as cell lines, for e.g. human cell lines. Alternatively, rat cells may be used.

According to a yet further aspect of the invention there is provided a host cell transformed or transfected with the vector of the invention.

According to a yet further aspect of the invention there is provided a recombinant cell line that is engineered to express a reporter molecule whose expression is under the control of the promoter of GH1 wherein said promoter comprises a plurality of the following SNP's: 1, 6, 7, 9, 11 or 14 and/or any one or more of the following haplotypes: 3, 4, 5, 7, 11, 13, 17, 19, 23, 24, 26 or 29 shown in Table 1.

According to a yet further aspect of the invention there is provided a transgenic non-human animal which under-expresses growth hormone as a result of having a GH1 promoter containing a plurality of the following SNP's: 1, 6, 7, 9, 11 and 14 and/or as a result of said promoter being characterised by one of the following haplotypes: 3, 4, 5, 7, 11, 13, 17, 19, 23, 24, 26 or 29, shown in Table 1.

In a preferred transgenic non-human animal of the invention said promoter is characterised by haplotype 23 or 27 and thus is termed a “low expressing promoter haplotype” or a “high expressing promoter haplotype”, respectively. These two haplotypes can be usefully used to compare and contrast the affects of candidate drugs on the growth patterns of said animals. Additionally, haplotype H1, in Table 1, may conveniently be used as a “normal expressing promoter haplotype”.

In a preferred embodiment of the invention said promoter is artificially engineered so as to be super-maximal expressing and its characterised by the haplotype AGGGGTTAT-ATGGAG or a sub-minimal promoter haplotype characterised by the sequence AG-TTGTGGGACCACT and AG-TTTTGGGGCCACT.

According to a further aspect of the invention there is therefore provided a method for screening for therapeutically active drugs which can be used to treat growth hormone dysfunction comprising exposing the cell, or cell line, of the invention to a candidate drug and then determining if the candidate drug has affected the activity of the promoter region of the growth hormone gene and so, in the case of the cell line, the expression of the reporter molecule.

According to a yet further aspect of the invention there is provided a method for screening for therapeutically active drugs which can be used to treat growth hormone dysfunction comprising exposing a transgenic non-human animal of the invention to candidate drugs and then monitoring the growth of said animal and where the candidate drug is shown to have a positive effect, in terms of animal growth, concluding that said growth is indicative of the therapeutic activity of said candidate drug.

Reference herein to a positive effect will most typically mean an ability to promote growth, however, in certain circumstances where a high expressing promoter is used the ability to affect growth may include an ability to inhibit growth.

The invention will now be exemplified with reference to the following materials and methods section.

Human Subjects

DNA samples were obtained from lymphocytes taken from 154 male British army recruits of Caucasian origin who were unselected for height. Height data were available for 124 of these individuals (mean, 1.76±0.07 m) and the height distribution was found to be normal (Shapiro-Wilk statistic W=0.984, p=0.16). Ethical approval for these studies was obtained from the local Multi-Regional Ethics Committee.

Polymerase Chain Reaction (PCR) Amplification

PCR amplification of a 3.2 kb GH1 gene-specific fragment was performed using oligonucleotide primers GH1F (5′ GGGAGCCCCAGCMTGC 3′; −615 to −599) and GH1R (5′ TGTAGGMGTCTGGGGTGC 3′; 2598 to 2616) [numbering relative to the transcriptional initiation site at +1 (GenBank Accession No. J03071)]. A 1.9 kb fragment containing sites I and II of the GH1 LCR was PCR amplified with LCR5A (5′ CCAAGTACCTCAGATGCAAGG 3′; −315 to −334) and LCR3.0 (5′ CCTTAGATCTTGGCCTAGGCC 3′; 1589 to 1698) [LCR sequence was obtained from GenBank (Accession No. AC005803) whilst LCR numbering follows that of Jin et al., Mol. Endocrinol. 13:1249-1266, 1999; GenBank (Accession No. AF010280)]. Conditions for both reactions were identical; briefly, 200 ng lymphocyte DNA was amplified using the Expand™ high fidelity system (Roche) using a hot start of 98° C. 2 min, followed by 95° C. 3 min, 30 cycles 95° C. 30 s, 64° C. 30 s, 68° C. 1 min. For the last 20 cycles, the elongation step at 68° C. was increased by 5 s per cycle. This was followed by further incubation at 68° C. for 7 min.

Cloning and Sequencing

Initially, PCR products were sequenced directly without cloning. The proximal promoter region of the GH1 gene was sequenced from the 3.2 kb GH1-specific PCR fragment using primer GH1S1 (5′ GTGGTCAGTGTTGGMCTGC 3′: −556 to −537). The 1.9 kb GH1 LCR fragment was sequenced using primers LCR5.0 (5′ CCTGTCACCTGAGGATGGG 3′; 993 to 101 1), LCR3.1 (5′ TGTGTTGCCTGGACCCTG 3′; 1093 to 1110), LCR3.2 (5′ CAGGAGGCCTCACAAGCC 3′; 628 to 645) and LCR3.3 (5′ ATGCATCAGGGCAATCGC 3′; 211 to 228). Sequencing was performed using BigDye v2.0 (Applied Biosystems) and an ABI Prism 377 or 3100 DNA sequencer. In the case of heterozygotes for promoter region or LCR variants, the appropriate fragment was cloned into pGEM-T (Promega) prior to sequencing.

Construction of Luciferase Reporter Gene Expression Vectors

Individual examples of 40 different GH1 proximal promoter haplotypes (Table 1) were PCR amplified as 582 bp fragments with primers GHPROM5 (5′ AGATCTGACCCAGGAGTCCTCAGC 3′; −520 to −501) and either GHPROM3A (5′ AAGCTTGCAGCTAGGTGAGCTGTC 3′; 44 to 62) or GHPROM3C (5′ AAGCTTGCCGCTAGGTGAGCTGTC 3′; 44 to 62) depending on the base at position +59 of the haplotype. To facilitate cloning, all primers had partial or complete non-templated restriction endonuclease recognition sequences added to their 5′ ends (underlined above); BglII (GHPROM5) and HindIII (GHPROM3A and GHPROM3C). PCR fragments were then cloned into pGEM-T. Plasmid DNA was initially digested with HindIII (New England Biolabs) and the 5′ overhang removed with mung bean nuclease (New England Biolabs). The promoter fragment was released by digestion with BglII (New England Biolabs) and gel purified. The luciferase reporter vector pGL3 Basic was prepared by Ncol (New England Biolabs) digestion and the 5′ overhang removed with mung bean nuclease. The vector was then digested with BglII (New England Biolabs) and gel purified. The restricted promoter fragments were cloned into luciferase reporter gene vector GL3 Basic. Plasmid DNAs (pGL3GH series) were isolated (Qiagen midiprep system) and sequenced using primers RV3 (5′ CTAGCAAAATAGGCTGTCCC 3′; 4760 to 4779), GH1SEQ1 (5′ CCACTCAGGGTCCTGTG 3′; 27 to 43), LUCSEQ1 (5′ CTGGATCTACTGGTCTGC 3′; 683 to 700) and LUCSEQ2 (5′ GACGAACACTTCTTCATCG 3′; 1372 to 1390) to ensure that both the GH1 promoter and luciferase gene sequences were correct. A truncated GH1 proximal promoter construct (−288 to +62) was also made by restriction of pGL3GH1 (haplotype 1) with Ncol and BglII followed by blunt-ending/religation to remove SNP sites 1-5.

Artificial proximal promoter haplotype reporter gene constructs were made by site-directed mutagenesis (SDM) [Site-Directed Mutagenesis Kit (Stratagene)] to generate the predicted super-maximal haplotype (AGGGGTTAT-ATGGAG) and sub-minimal haplotypes (AG-TTGTGGGACCACT and AG-TTTTGGGGCCACT).

To make the LCR-proximal promoter fusion constructs, the 1.9 kb LCR fragment was restricted with BglII and the resulting 1.6 kb fragment cloned into the BglII site directly upstream of the 582 bp promoter fragment in pGL3. The three different LCR haplotypes were cloned in pGL3 Basic, 5′ to one of three GH1 proximal promoter constructs containing respectively a “high expressing promoter haplotype” (H27), a “low expressing promoter haplotype” (H23) and a “normal expressing promoter haplotype” (H1) to yield a total of nine different LCR-GH1 proximal promoter constructs (pGL3GHLCR). Plasmid DNAs were then isolated (Qiagen midiprep) and sequence checked using appropriate primers.

Luciferase Reporter Gene Assays

In the absence of a human pituitary cell line expressing growth hormone, rat GC pituitary cells (Bancroft, Endocrinology 92:1014-1021, 1973; Bodner and Karin, Cell 50:267-275, 1989) were selected for in vitro expression experiments. Rat GC cells were grown in DMEM containing 15% horse serum and 2.5% fetal calf serum. Human HeLa cells were grown in DMEM containing 5% fetal calf serum. Both cell lines were grown at 37° C. in 5% CO₂. Liposome-mediated transfection of GC cells and HeLa cells was performed using Tfx™-20 (Promega) in a 96-well plate format. Confluent cells were removed from culture flasks, diluted with fresh medium and plated out into 96-well plates so as to be ˜80% confluent by the following day.

The transfection mixture contained serum-free medium, 250 ng pGL3GH or pGL3GHLCR construct, 2ng pRL-CMV, and 0.5 μl Tfx™-20 Reagent (Promega) in a total volume of 90 μl per well. After 1 hr, 200 μl complete medium was added to each well. Following transfection, the cells were incubated for 24 hrs at 37° C. in 5% CO₂ before being lysed for the reporter assay.

Luciferase assays were performed using the Dual Luciferase Reporter Assay System (Promega). Assays were performed on a microplate luminometer (Applied Biosystems) and then normalized with respect to Renilla activity. Each construct was analysed on three independent plates with six replicates per plate (i.e. a total of 18 independent measurements). For the proximal promoter assays, each plate included negative (promoterless pGL3 Basic) and positive (SV40 promoter-containing pGL3) controls. For the LCR analysis, constructs containing the proximal promoter but lacking the LCR were used as negative controls.

Electrophoretic Mobility Shift Assay (EMSA)

EMSA was performed on double stranded oligonucleotides that together covered all 16 SNP sites (see Supplementary Material Online). Nuclear extracts from GC and HeLa cells were prepared as described by Berg et al. (Berg et al., Hum. Mol. Genet. 3:2147-2152, 1994). Oligonucleotides were radiolabelled with [γ-³³P]-dATP and detected by autoradiography after gel electrophoresis. EMSA reactions contained a final concentration of 20 mM Hepes pH7.9, 4% glycerol, 1 mM MgCl₂, 0.5 mM DTT, 50 mM KCl, 1.2 μg HeLa cell or GC cell nuclear extract, 0.4 μg poly[dl-dC].poly[dl-dC], 0.4 pM radiolabelled oligonucleotide, 40 pM unlabelled competitor oligonucleotide (100-fold excess) where appropriate, in a final volume of 10 μl. EMSA reactions were incubated on ice for 60 mins and electrophoresed on 4% PAGE gels at 100V for 45 mins prior to autoradiography. For each reaction, a double stranded unlabelled test oligonucleotide was used as a specific competitor whilst an oligonucleotide derived from the NF1 gene promoter (5′ CCCCGGCCGTGGAAAGGATCCCAC 3′) was used as a non-specific competitor. Double stranded oligonucleotides corresponding to the human prolactin (PRL) gene Pit-1 binding site (5′ TCATTATATTCATGMGAT 3′) and the Pit-1 consensus binding site (5′ TGTCTTCCTGAATATGMTMGAAATA 3′) were used as specific competitors for protein binding to the SNP 8 site.

Primer Extension Assays

Primer extension assays were performed to confirm that constructs bearing different SNP haplotypes utilized identical transcriptional initiation sites. Primer extension followed the method of Triezenberg et al. (Triezenberg et al., Primer Extension, Current Protocols in Molecular Biology, New York, John Wiley, p. 4.8.1-4.8.5, 1992).

Data Normalization

Expression measurements for negative controls (promoterless pGL3 Basic) exhibited considerable variation between plates. To correct the data for base-line expression and plate effects, the mean activity of the negative controls on a given plate was subtracted from all other activity values on the same plate. The mean (plate-corrected) activity for the wild-type proximal promoter haplotype 1 (H1) on each plate was then calculated, and all other haplotype-associated activities on the same plate were divided by this value. These two transformations ensured that the mean negative control activity equalled zero whilst the mean activity of H1 equalled unity, independent of plate number. Resulting activity values may thus be interpreted as fold changes in comparison to H1, corrected for both baseline and plate effects. Since no significant plate effect was detectable after transformation, the data were combined over plates. A similar procedure was also followed for the LCR-promoter fusion construct expression data, using haplotype A as the reference haplotype.

Statistical Analysis

Normalized expression levels of the proximal promoter haplotypes were tested for goodness-of-fit to a Gaussian distribution using the Shapiro-Wilk statistic (W) as implemented in procedure UNIVARIATE of the SAS statistical analysis software (SAS Institute Inc., Cary N.C., USA). Significance assessment was adjusted for multiple (i.e. 40-fold) testing by setting p_{critical}=0.05/40≈0.001. Using this criterion, the expression levels of two promoter haplotypes were found to differ significantly from a Gaussian distribution viz. H21 (W=0.727, p=0.0002) and H40 (W=0.758, p=0.0004). For the other 38 haplotypes, expression levels were regarded as consistent with normality and therefore subjected to pair-wise comparison using Tukey's studentized range test (SAS procedure GLM). Pair-wise comparison of expression levels between groups of different haplotypes was performed using normal approximation z of the Wilcoxon rank sum statistic (SAS procedure NPAR1WAY).

In order to assess formally the correlation structure between the SNPs, and to be able to identify an appropriate subset of critical polymorphisms for further study, the residual deviance upon haplotype partitioning was calculated for all possible subsets of proximal promoter SNPs.

For a given partitioning {1 . . . M}=π=π₁∪ . . . ∪πₖ of a set of data points x1, . . . , xₘ, and with π(i)=j if i∈πⱼ, the residual deviance δ of π is defined as

$$δ = δ ( Π ) = {\sum }_{i = 1}^{m} {( {x}_{i} - {\overline{x}}_{π ( i )} )}^{2} .$$

When the dataset was not partitioned at all, then δ=δ(π₀)=421.7, and the relative residual deviance of any other partitioning π was defined as δ_{R}(π)=δ(π)/δ(π₀).

Six SNPs (nos. 1, 6, 7, 9, 11 and 14; see below) were identified as being responsible for a sizeable proportion (˜60%) of the residual deviance in expression level at the same time as invoking relatively little haplotype variation. The statistical interdependence of these SNPs was further analysed by means of a regression tree, constructed by recursive binary partitioning using statistics software R (Ihaka and Gentleman, J. Comput. Graph. Stat. 5:299-314, 1996). In the tree construction process, the SNPs were used individually as predictor variables at each node so as to select the two most homogeneous subgroups of haplotypes with respect to the response variable (i.e. normalized proximal promoter expression). The node and SNP that served to introduce a new split were chosen so as to minimize δ_{R} for the partitioning as defined by the terminating nodes (‘leafs’) of the resulting intermediate tree. This process was continued until all leafs corresponded to individual haplotypes (‘fully grown tree’). The reliability of the δ_{R} estimates was assessed in each step by 10-fold cross-validation and the standard error (SE) was calculated.

Regression analysis of height and proximal promoter expression level in vitro was performed for the 124 height-known individuals studied using the REG procedure of the SAS software package. Let μ_{nor,h1} and μ_{nor,h2} denote the mean normalized expression levels of the two haplotypes carried by a given individual. The height of individuals not homozygous for H1 (n=109) was modelled as

$$height = {a}_{0} + {a}_{1} \cdot \frac{{μ}_{nor , h1} + {μ}_{nor , h2}}{2} + {a}_{2} \cdot \frac{{μ}_{nor , h1}^{2} + {μ}_{nor , h2}^{2}}{2} + {a}_{3} \cdot {μ}_{nor , h1} \cdot {μ}_{nor , h2}$$

and the coefficient of determination, r², calculated.

A reduced median network (Bandelt et al., Genetics 141:743-753, 1995) was constructed for the seven promoter haplotypes (H1-H7) that were observed at least 8 times in the 154 study individuals.

Linkage Disequilibrium Analysis

Linkage disequilibrium (LD) between promoter SNPs, and between individual SNPs and the LCR haplotypes, was evaluated in 100 individuals randomly chosen from the total of 154 under study, using parameter ρ as devised for biallelic loci by Morton et al. (Morton et al., Proc. Natl. Acad. Sci. USA 98:5217-5221, 2001). Whilst ρ=1 is equivalent to two loci showing complete LD, ρ=0 indicates complete lack of LD. Only eight SNPs were found to be sufficiently polymorphic in the population sample (heterozygosity ≧5%) to warrant inclusion. SNP5 was excluded owing to its perfect LD with SNP4 (only two pair-wise haplotypes present). Maximum likelihood estimates of the combined LCR-proximal promoter haplotype frequencies, as required for LD analysis, were obtained using an in-house implementation of the expectation maximization (EM) algorithm.

Results

Proximal Promoter Haplotypes and Relative Promoter Strength

The 40 promoter haplotypes were studied by in vitro reporter gene assay and found to differ with respect to their ability to drive luciferase gene expression in rat pituitary cells (Table 3). Expression levels were found to vary over a 12-fold range with the lowest expressing haplotype (no. 17) exhibiting an average level that was 30% that of wild-type and the highest expressing haplotype (no. 27) exhibiting an average level that was 389% that of wild-type (Table 3). Twelve haplotypes (nos. 3, 4, 5, 7, 11, 13, 17, 19, 23, 24, 26 and 29) were associated with a significantly reduced level of luciferase reporter gene expression by comparison with H1. Conversely, a total of 10 haplotypes (nos. 14, 20, 27, 30, 34, 36, 37, 38, 39 and 40) were associated with a significantly increased level of luciferase reporter gene expression by comparison with H1 (Table 3). Constructs bearing different SNP haplotypes were shown by primer extension assay to utilize identical transcriptional initiation sites (data not shown). Expression of the reporter gene constructs was found to be 1000-fold lower in HeLa cells than in GC cells (data not shown).

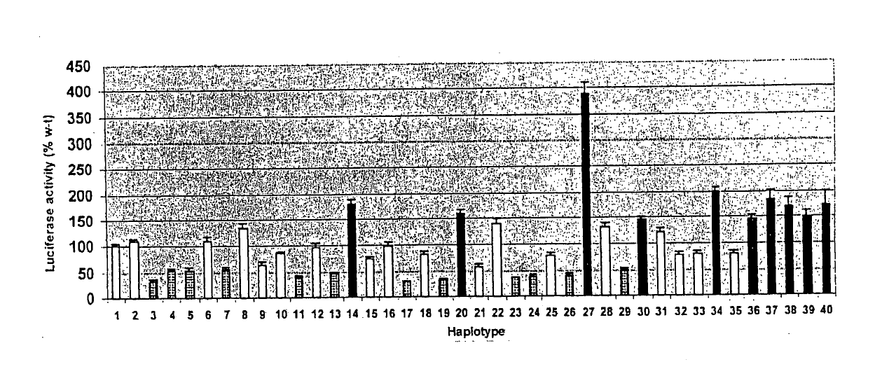

The in vitro expression levels of the 40 different GH1 promoter haplotypes are presented graphically in FIG. 2. A significant trend is apparent for the low expressing haplotypes to occur more frequently whereas the high expressing haplotypes tend to occur less frequently (Wilcoxon p<0.01). Since this finding is suggestive of the action of selection, selection effects were sought at the level of individual SNPs. For the 15 SNPs studied here, the mean expression level (weighted by haplotype frequency) and the frequency of the rarer allele in controls were found to be positively correlated (Spearman rank correlation coefficient, r=0.32, one-sided p<0.10). If SNP 7 is excluded as an obvious outlier (it has a particularly high expression level associated with the rarer allele), r=0.53 with a one-sided p<0.05.

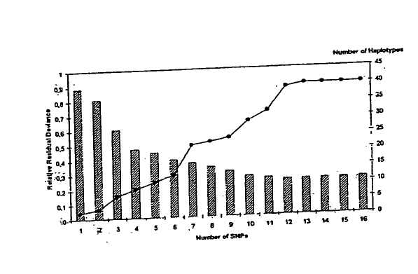

Expression levels associated with individual SNPs were found to be strongly interdependent. An attempt was therefore made to partition the expression data in such a way as to identify a subset of key polymorphic sites that contribute disproportionately to the observed variation in in vitro expression level. Partitioning by the full haplotype comprising all 16 SNPs yielded a relative residual deviance of δ_{R}(π₁₆)=0.245. This can be interpreted in terms of 24.5% of the variation in expression level not being accountable by variation in haplotype. For 1≦k<16, the minimum-δ_{R}-partitioning π_{k,min} was defined as that haplotype partitioning with k SNPs that yielded the smallest relative residual deviance δ_{R}. The relationship between k and δ_{R}(π_{k,min}), together with the number of haplotypes comprising π_{k,min}, is depicted in FIG. 3. A qualitative difference was evident between k=6 and k=7 in that the number of haplotypes associated with π_{k,min} increases from 13 to 22 whilst δ_{R}(π_{k,min}) decreases only marginally [δ_{R}(π_{6,min})=0.397 vs δ_{R}(π_{7,min})=0.371]. It was therefore concluded that SNPs 1, 6, 7, 9, 11 and 14, which define π_{6,min}, represented a good choice of key polymorphisms for further analysis. Of the remaining SNPs, six (nos. 3, 4, 8, 10, 12, and 16) could be classified as “marginally informative”. These markers, in combination with the six key SNPS, together define 39 of the 40 haplotypes observed, and account for virtually all of the explicable deviance (δ_{R}(π_{12,min})=0.245). The other four SNPs (nos. 2, 5, 13 and 15) were “uninformative” with respect to the normalized in vitro expression level since they were either monomorphic in our sample (no. 2), or were in perfect (nos. 5 and 13) or near perfect (no. 15) linkage disequilibrium with other markers.

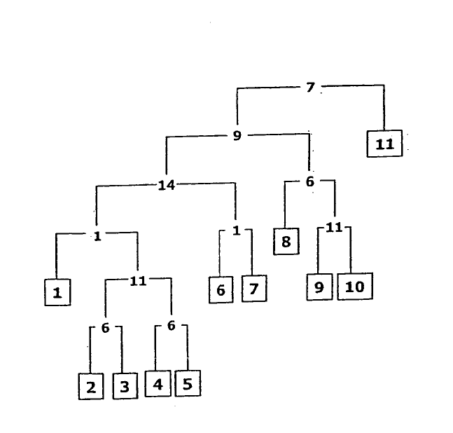

The correlation structure of the six key SNPs was next assessed using a series of successively growing (i.e. nested) regression trees. Following convention in regression tree analysis (Therneau and Atkinson, An Introduction to Recursive Partitioning Using RPART Routines, Technical Report #61, Rochester, Minn.: The Mayo Foundation, p. 309-310,1997), the smallest intermediate tree with a cross-validated δ_{R} within one SE of that of the fully grown tree was chosen as a representative partitioning. This ‘optimal’ tree was found to comprise 10 internal and 11 terminal nodes (FIG. 4, Table 4). The relative residual deviance of the tree equals δ_{R}=0.398, thereby accounting for (1-0.397)/(1-0.245)≈80% of the deviance explicable through haplotype partitioning.

The single most important split was by SNP 7 which on its own accounted for 15% of the explicable deviance. The four haplotypes carrying the C allele of this SNP define a homogeneous subgroup (leaf 11) with a mean normalized expression level 1.8 times higher than that of H1. Haplotypes carrying the T allele of SNP 7 were further sub-divided by SNP 9, with allele T of this polymorphism causing higher expression (μₙₒᵣ=1.26) than allele G (μₙₒᵣ=0.84; Wilcoxon z=7.09, p<0.001). The resulting nnTTnn haplotype was split by SNP 6 (G/T), with nGTTnn forming a terminal node (leaf 8) that includes the wild-type haplotype H1. Interestingly, the nTTTnn haplotypes, when sub-divided by SNP 11, manifested a dramatic difference in expression level. Whilst nTTTGn (leaf 9) was found to be a low expresser (μₙₒᵣ=0.64), haplotype nTTTAn (leaf 10) exhibited maximum average expression (μₙₒᵣ=3.89; Wilcoxon z=5.11, p<0.001).

Haplotype nnTGnn for SNPs 7 and 9 was sub-divided by SNPs 14 and 1, with three of the resulting haplotypes forming terminal nodes (leafs 1, 6 and 7). The fourth haplotype, GnTGnA, was an intermediate expresser (μₙₒᵣ=0.86) that was further split by SNPs 11 and 6. Interestingly, only one particular combination of SNP 14 and 1 alleles resulted in increased expression on the SNP 7 and 9 nnTGnn background (AnTGnG, leaf 7, μₙₒᵣ=1.83). A similar non-additive effect upon expression was also noted for SNPs 6 and 11 when considered on haplotype GnTGnA: whereas SNP 11 allele A was associated with higher expression than G in combination with SNP 6 allele T (GTTGAA, leaf 5, μₙₒᵣ=1.18 vs GTTGGA, leaf 2, μₙₒᵣ=0.74; Wilcoxon z=7.09, p<0.001), the opposite held true in combination with SNP 6 allele G (GGTGAA, leaf 4, μₙₒᵣ=0.74 vs GGTGGA, leaf 3, μₙₒᵣ=1.04; Wilcoxon z=5.28, p<0.001).

Evolution of Haplotype Diversity

Of the 15 GH1 gene promoter SNPs found to be polymorphic in this study, alternative alleles at 14 positions were potentially explicable by gene conversion since they were identical to those in analogous locations in at least one of the four paralogous human genes (Table 2). Comparison with the orthologous GH gene promoter sequences of 10 other mammals revealed that the most frequent alleles at nucleotide positions −75, −57, −31, −6, +3, +16 and +25 (corresponding to SNPs 8-15 inclusive) in the human GH1 gene were strictly conserved during mammalian evolution (Krawczak et al., Gene 237:143-151, 1999). Intriguingly, the rarest of the three alternative alleles at the −1 position (SNP 12) in the human GH1 gene was identical to that strictly conserved in the mammalian orthologues.

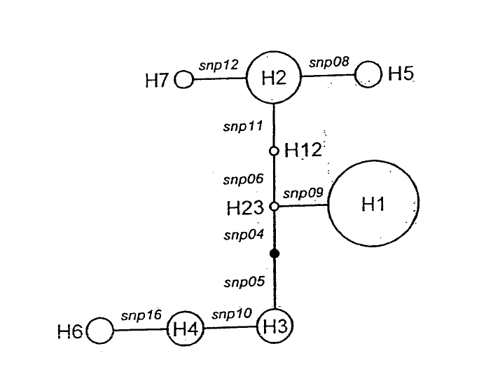

A ‘Reduced Median Network’ (FIG. 5) revealed that wild-type haplotype H1 is not directly connected to other frequent haplotypes by single mutational events. The second most common haplotype, H2, is connected to H1 via H23 and H12 whilst the third most common haplotype, H3, is connected to H1 either through a non-observed haplotype or a double mutation. Expansion of this network so as to incorporate further haplotypes was deemed unreliable owing to the small number of observations per haplotype. Furthermore, expansion of the network would have entailed the introduction of multiple single base-pair substitutions. Since these cannot be distinguished from serial rounds of gene conversion between pre-existing haplotypes, the resulting distances in the network would have been unlikely to reflect genuine evolutionary relationships. However, this may safely be assumed to be the case for the network depicted in FIG. 5 that connects the seven most frequent haplotypes, since each mutation occurs only once.

A general decline of linkage disequilibrium (LD) with physical distance was noted for most SNPs, with some notable exceptions (Table 5). Thus, SNP 9 was found to be in strong LD with the other SNPs, including SNP 16 which showed comparatively weak LD with all other proximal promoter SNPs. This finding suggests that the origin of SNP 9 was relatively late. However, SNP 10 was found to be in perfect LD with SNP 12 but not SNP 11 (p=0.381), whereas SNP 8 was in stronger LD with SNP 11 than with SNP 10 (p=0.925 vs 0.687). These anomalous findings suggest that the extant pattern of LD among the proximal promoter SNPs is unlikely to have arisen solely through recombinational decay with distance, but rather is likely to reflect the action of other mechanisms such as recurrent mutation, gene conversion or selection.

Prediction and Functional Testing of Super-Maximal and Sub-Minimal Haplotypes

Based upon the ‘optimal’ regression tree obtained for the haplotype-dependent proximal promoter expression data, an attempt was made to predict potential “super-maximal” and “sub-minimal” haplotypes in terms of their levels of expression. To this end, alleles of the six key SNPs were chosen taking the mean expression levels of the appropriate leafs of the tree into account (Table 4). Alleles of the remaining SNPs were determined so as to respectively maximize or minimize expression of individual SNPs. Thus, for the predicted super-maximal haplotype, alleles of SNPs 6, 7, 9 and 11 were as in leaf 10 whilst alleles of SNPs 1 and 14 were as in leaf 7. The sub-minimal haplotype was chosen to represent leaf 1 (for SNPs 1, 7, 9 and 14). The best choice of alleles for SNPs 6 and 11 was however somewhat ambiguous since leafs 2 (suggesting alleles T and G) and 4 (suggesting alleles G and A) predicted similarly low mean expression levels. Therefore, it was decided to generate both constructs for in vitro testing. Completion of the hypothetical haplotypes for the remaining SNPs yielded super-maximal haplotype AGGGGTTAT-ATGGAG and sub-minimal haplotypes AG-TTGTGGGACCACT and AG-TTTTGGGGCCACT.

These three artificial haplotypes were then constructed and expressed in rat pituitary cells yielding respectively expression levels of 145±4, 55±5 and 20±8% in comparison to wild-type (haplotype 1).

Differences Between SNP Alleles Revealed by Mobility Shift (EMSA) Assay

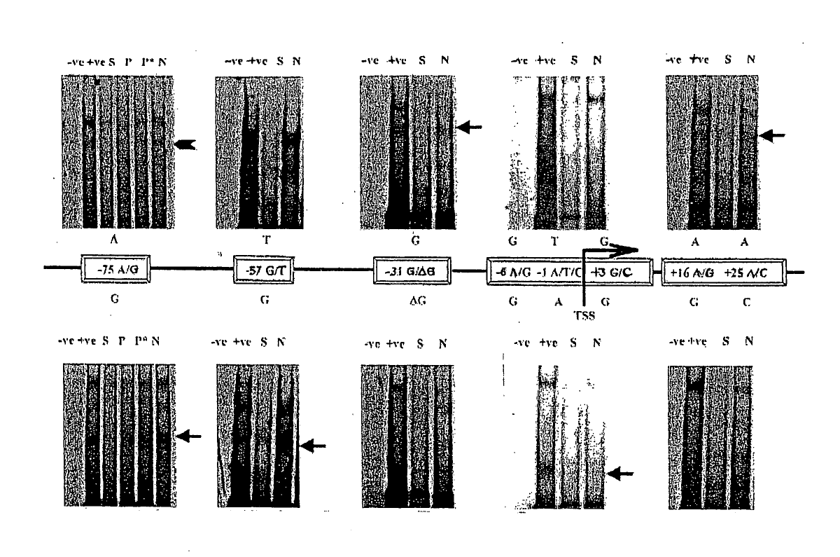

EMSAs were performed at all proximal promoter SNP sites for all allelic variants using rat pituitary cells as a source of nuclear protein. Protein interacting bands were noted at sites −168, −75, −57, −31, −6/−1/+3 and +16/+25 (Table 6). Inter-allelic differences in the number of protein interacting bands were noted for sites −75 (SNP 8), −57 (SNP 9), −31 (SNP 10), −6/−1/+3 (SNPs 11, 12, 13) and +16/+25 (SNPs 14, 15) [FIG. 6; Table 6]. In the case of the latter two sites, EMSA assays on specific SNP allele combinations suggested that differential protein binding was attributable to allelic variation at SNP sites 12 and 15 respectively (Table 6). When the analysis was repeated using a HeLa cell extract, only position −57 manifested evidence of a protein interaction and then only for the G allele, not the T allele (data not shown). The results of competition experiments utilizing oligonucleotides corresponding to two distinct Pit-1 binding sites were consistent with one of the two SNP 8 interacting proteins being Pit-1 (FIG. 6). However, the allele-specific protein interaction remained unaffected implying that the other protein involved was not Pit-1.

Association Between Promoter Haplotype Expression in Vitro and Stature in Vivo

An attempt was made to correlate the haplotype-specific in vitro expression of the GH1 proximal promoter with adult height in 124 male Caucasians. Each haplotype was ascribed its mean expression value from normalized in vitro expression data (Table 3) and the average Aₓ=(μ_{nor,h1}+μ_{nor,h2})/2 of the two haplotypes was calculated for each individual. Individuals homozygous for H1 were excluded from the analysis since their Aₓ values (1.0) would not have contributed any causal variation. This yielded a sample of 109 height-known individuals with suitable genotypes (Table 7). When height above and below the median (1.765 m) was compared to Aₓ values above and below the median (0.9), evidence for an association between height and GH1 proximal promoter haplotype-associated in vitro expression emerged (χ²=4.846, 1 d.f., p=0.028). This notwithstanding, regression analysis using a 2^{nd} degree polynomial demonstrated that the two μₙₒᵣ values were on their own relatively poor predictors of height. Since the coefficient of determination was r²=0.033 (p>0.5), it may be concluded that approximately 3.3% of the variance in body height is accounted for by reference to GH1 gene proximal promoter haplotype expression in vitro.

Locus Control Region (LCR) Polymorphisms and Proximal Promoter Strength

Three novel polymorphic changes were found within sites I and II (required for the pituitary-specific expression of the GH1 gene; Jin et al., supra, 1999) of the GH1 LCR in a screen of 100 individuals randomly chosen from the study group. These were located at nucleotide positions 990 (G/A; 0.90/0.10), 1144 (A/C; 0.65/0.35) and 1194 (C/T; 0.65/0.35) [numbering after Jin et al., supra, 1999]. The polymorphisms at 1144 and 1194 were in total linkage disequilibrium, and three different haplotypes were observed: haplotype A (990G, 1144A, 1194C; 0.55), haplotype B (990G, 1144C, 1194T; 0.35) and haplotype C (990A, 1144A, 1194C; 0.10).

In order to determine whether the three LCR haplotypes exert a differential effect on the expression of the downstream GH1 gene, a number of different LCR-GH1 proximal promoter constructs were made. The three alternative 1.6 kb LCR-containing fragments were cloned into pGL3, directly upstream of three distinct types of proximal promoter haplotype, viz. a “high expressing promoter” (H27), a “low expressing promoter” (H23) and a “normal expressing promoter” (H1), to yield nine different LCR-GH1 proximal promoter constructs in all. These constructs were then expressed in both rat GC cells and HeLa cells, and the resulting luciferase activities measured. In GC cells, the presence of the LCR enhances expression up to 2.8-fold as compared to the proximal promoter alone (Table 8). However, the extent of this inductive effect was dependent upon the linked promoter haplotype. Two-way analysis of variance (Table 9) revealed that both main effects and the promoter*LCR interaction were significant (p<0.0001), with the major influence exerted by the proximal promoter. Also included in Table 8 are the results of a Tukey studentized range test at 95% significance level, performed individually for each promoter haplotype. In conjunction with promoter haplotype 1, the activity of LCR haplotype A is significantly different from that of N (construct containing proximal promoter but lacking LCR), but not from that of LCR haplotypes B and C; LCR haplotypes B and C differ significantly from each other and from N. With promoter 27, however, no significant difference was found between LCR haplotypes. No LCR-mediated induction of expression was noted with any of the proximal promoter haplotypes in HeLa cells (data not shown).

Since the physical distance between the LCR and the proximal promoter SNPs was too great to permit joint physical haplotyping, the linkage disequilbrium (LD) between them was assessed by maximum likelihood methods using genotype data from the 100 individuals included in the analysis of inter-SNP LD for the proximal promoter. Pair-wise LD between promoter SNPs and LCR haplotypes was found to be high for all SNPs except SNP 16 (Table 5). It may therefore be concluded that SNP 16 was subject to recurrent mutation prior to the genesis of SNP 9, the only SNP found to be in strong linkage disequilibrium with SNP 16. Substantial differences between LCR haplotypes exist in terms of their LD with SNPs 4, 8 and 16 (Table 5), suggesting a relatively young age for LCR haplotype B as opposed to haplotype A.

Conclusions

Partitioning of the haplotypes identified six SNPs (nos. 1, 6, 7, 9, 11 and 14) as major determinants of GH1 gene expression level, with a further six SNPs being marginally informative (nos. 3, 4, 8, 10, 12 and 16). The functional significance of all 16 SNPs was investigated by EMSA assays which indicated that six polymorphic sites in the GH1 proximal promoter interact with nucleic acid binding proteins; for five of these sites [−75 (SNP 8), −57 (SNP 9), −31 (SNP 10), −1 (SNP 12) and +25 (SNP 15)], alternative alleles exhibited differential protein binding. Of these five sites, only SNP 9 was also identified as a major determinant of GH1 gene expression level by recursive partitioning. This apparent discrepancy may be explicable in terms of regression tree analysis taking into account the full genetic variation manifest in all 40 haplotypes. Furthermore, in the partitioning procedure, individual SNPs are evaluated on the basis of their net effect upon expression level, and not through directly measurable functional characteristics. This implies that factors other than allele-specific protein binding may have played a role in determining the position of individual SNPs in the regression tree.

The molecular basis for haplotype-dependent differences in GH1 gene promoter strength may thus lie in the net effect of the differential binding of multiple transcription factors to alternative arrays of their cognate binding sites. These arrays differ by virtue of their containing different alleles of the various SNPs that combinatorially constitute the observed promoter haplotypes. Some transcription factors are coordinated directly by cis-acting DNA sequence motifs, others indirectly by protein-protein interactions in what has been likened to a three-dimensional jigsaw puzzle: the DNA sequence motifs providing the puzzle template, the transcription factors constituting the puzzle pieces. This modular view of the promoter helps one to envisage how the effect of different SNP combinations in a given haplotype might be transduced so as to exert differential effects on transcription factor binding, transcriptosome assembly and hence gene expression. Thus, for example, the observed non-additive effects of GH1 promoter SNPs on gene expression may be understood in terms of the allele-specific differential binding of a given protein at one SNP site affecting in turn the binding of a second protein at another SNP site that is itself subject to allele-specific protein binding.

The LCR upstream of the GH gene cluster contains sequence elements that possess enhancer activity, confer tissue specificity of expression, and promote long range gene activation through the spreading of histone acetylation (Shewchuk et al., J. Biol. Chem. 274:35725-35733, 1999; Su et al., J. Biol. Chem. 275:7902-7909, 2000; Shewchuk et al., Nucleic Acids Res. 29:3356-3361, 2001; Ho et al., Mol. Cell 9:291-302, 2002). The somatotrope-specific determinants of the LCR are present within a 1.6 kb region (sites I and II) ˜14.5 kb upstream of the GH1 gene (Shewchuk et al., supra, 1999). In our own system, the introduction of this 1.6 kb LCR fragment served to enhance the activity of the GH1 proximal promoter by up to 2.8-fold, although the degree of enhancement was found to be dependent upon the identity of the linked proximal promoter haplotype. Conversely, enhancement of the activity of a proximal promoter of given haplotype was also found to be dependent upon the identity of the LCR haplotype. Taken together, these findings imply that the genetic basis of inter-individual differences in GH1 gene expression is likely to be extremely complex.

TABLE 1
GH1 proximal promoter haplotypes defined by genetic variation at 16 locations.

| | SNP position relative to GH1 gene transcriptional start site | | | | | | | | | | | | | | | | |
|---|---|---|---|---|---|---|---|---|---|---|---|---|---|---|---|---|---|
| No. n | −476 | −364 | −339 | −308 | −301 | −278 | −168 | −75 | −57 | −31 | −6 | −1 | +3 | +6 | +25 | +59 | |
| 1 | G | G | G | G | G | G | T | A | T | G | A | A | G | A | A | T | 103 |
| 2 | G | G | G | G | G | T | T | A | G | G | G | A | G | A | A | T | 50 |
| 3^{§} | G | G | G | T | T | G | T | A | G | G | A | A | G | A | A | T | 28 |
| 4^{§} | G | G | G | T | T | G | T | A | G | — | A | A | G | A | A | T | 16 |
| 5^{§} | G | G | G | G | G | T | T | G | G | G | G | A | G | A | A | T | 13 |
| 6 | G | G | G | T | T | G | T | A | G | — | A | A | G | A | A | G | 9 |
| 7^{§} | G | G | G | G | G | T | T | A | G | G | G | T | G | A | A | T | 8 |
| 8 | G | G | G | T | T | G | T | A | G | G | G | A | G | A | A | T | 6 |
| 9 | G | G | G | G | G | T | T | A | T | G | G | A | G | A | A | T | 6 |
| 10 | G | G | G | T | T | G | T | A | G | — | G | A | G | A | A | T | 6 |
| 11^{§} | G | G | G | G | G | T | T | G | G | G | G | A | G | G | C | T | 5 |
| 12 | G | G | G | G | G | T | T | A | G | G | A | A | G | A | A | T | 5 |
| 13^{§} | G | G | — | G | G | T | T | G | G | G | G | A | G | A | A | T | 5 |
| 14 | G | G | G | G | G | T | C | A | G | G | G | T | G | A | A | T | 5 |
| 15 | G | G | G | T | T | G | T | A | G | G | G | T | G | A | A | T | 4 |
| 16 | G | G | G | G | G | T | T | G | G | G | A | A | G | A | A | T | 4 |
| 17^{§} | G | G | — | G | G | T | T | A | G | G | G | A | G | A | A | T | 4 |
| 18 | G | G | G | G | G | T | T | A | G | — | G | A | G | A | A | T | 3 |
| 19^{§} | A | G | G | G | G | T | T | A | G | G | G | A | G | A | A | T | 3 |
| 20 | G | G | G | G | G | G | T | A | G | — | A | A | G | A | A | T | 3 |
| 21 | G | G | G | G | G | T | T | G | G | G | G | A | G | A | A | G | 3 |
| 22 | G | G | G | T | T | G | T | A | T | G | A | A | G | A | A | T | 3 |
| 23^{§} | G | G | G | G | G | G | T | A | G | G | A | A | G | A | A | T | 2 |
| 24^{§} | G | G | G | T | T | G | T | G | G | — | A | A | G | A | A | T | 2 |
| 25 | G | G | G | T | T | G | T | A | G | G | A | A | G | A | A | G | 1 |
| 26^{§} | G | G | G | G | G | T | T | G | G | G | G | T | G | A | A | T | 1 |
| 27 | G | G | G | G | G | T | T | A | T | G | A | A | G | A | A | T | 1 |
| 28 | G | G | G | G | G | T | T | A | G | — | A | A | G | A | A | T | 1 |
| 29^{§} | A | G | G | G | G | T | T | A | G | G | A | A | G | A | A | T | 1 |
| 30 | G | G | — | G | G | T | T | A | G | G | A | A | G | A | A | T | 1 |
| 31 | G | G | G | G | G | T | T | G | G | — | G | A | G | A | A | T | 1 |
| 32 | G | G | G | T | T | G | T | G | G | G | G | A | G | A | A | G | 1 |
| 33 | G | G | G | G | G | T | T | A | G | G | G | A | G | G | C | T | 1 |
| 34 | G | G | — | G | G | T | C | A | G | G | G | T | G | A | A | T | 1 |
| 35 | G | G | G | G | G | G | T | A | G | G | A | C | G | A | A | T | 1 |
| 36 | G | G | G | G | G | T | T | A | G | G | G | T | G | A | A | G | 1 |
| 37^{§} | A | G | G | G | G | T | T | A | G | G | G | A | G | G | A | T | 0 |
| 38^{§} | G | G | G | G | G | T | C | A | G | G | A | A | G | A | A | T | 0 |
| 39^{§} | G | G | G | T | T | G | T | A | G | G | G | A | G | A | C | T | 0 |
| 40^{§} | G | G | G | G | G | T | C | A | G | G | G | A | G | A | A | T | 0 |

n: frequency in 154 male British Caucasioans;
^{§}haplotyps exhibiting a significatly reduced level (≦55% that of haplotype 1) of luciferase activity in GC cells;
^{$}only found in solitary cases of GH deficiency.
— denotes the absence of the base in question.

TABLE 2
Allele frequencies of 15 SNPs in the GH1
gene promoter of 154 male Caucasians and
corresponding nucleotides in analogous locations
of the paralogous genes of the GH cluster

| | Posi- | GH1 | | GH1 paralogues^{§} | | | |
|---|---|---|---|---|---|---|---|
| SNP | tion^{$} | Allele | Frequency | GH2 | CSH1 | CSH2 | CSHP1 |
| 1 | −476 | G | 304 (0.987) | A | G | G | A |
| | | A | 4 (0.013) | | | | |
| 3 | −339 | G | 297 (0.964) | G | G | G | G |
| | | — | 11 (0.036) | | | | |
| 4 | −308 | G | 232 (0.753) | T | C | C | T |
| | | T | 76 (0.247) | | | | |
| 5 | −301 | G | 232 (0.753) | T | T | T | T |
| | | T | 76 (0.247) | | | | |
| 6 | −278 | G | 185 (0.601) | T | A | A | T |
| | | T | 123 (0.399) | | | | |
| 7 | −168 | T | 302 (0.981) | T | C | C | T |
| | | C | 6 (0.019) | | | | |
| 8 | −75 | A | 273 (0.886) | G | A | A | G |
| | | G | 35 (0.114) | | | | |
| 9 | −57 | G | 195 (0.633) | A | T | T | G |
| | | T | 113 (0.367) | | | | |
| 10 | −31 | G | 267 (0.867) | — | G | G | G |
| | | — | 41 (0.133) | | | | |
| 11 | −6 | A | 181 (0.588) | A | G | G | A |
| | | G | 127 (0.412) | | | | |
| 12 | −1 | A | 287 (0.932) | A | T | T | C |
| | | T | 20 (0.065) | | | | |
| | | C | 1 (0.003) | | | | |
| 13 | +3 | G | 307 (0.997) | G | G | G | C |
| | | C | 1 (0.003) | | | | |
| 14 | +16 | A | 302 (0.981) | A | A | A | G |
| | | G | 6 (0.019) | | | | |
| 15 | +25 | A | 302 (0.981) | A | A | A | C |
| | | C | 6 (0.019) | | | | |
| 16 | +59 | T | 293 (0.951) | G | G | G | G |
| | | G | 15 (0.049) | | | | |

^{$}relative to the GH1 transcription start site;
^{§}bases at the analogous positions in the wild-type sequences of the four paralogous genes in the human GH cluster.

TABLE 3!In vitro GHJ gene promoter expression analysis? !of 40 different SNP haplotypes? !Haplotype No.? n? μₙₒᵣ? σₙₒᵣ? Tukey

| | | | | |
|---|---|---|---|---|
| 17 | 18 | 0.304 | 0.054 | a---------------- |
| 3 | 18 | 0.324 | 0.170 | a---------------- |
| 19 | 18 | 0.332 | 0.062 | a---------------- |
| 23 | 18 | 0.359 | 0.042 | ab--------------- |
| 24 | 18 | 0.395 | 0.107 | abc-------------- |
| 11 | 18 | 0.406 | 0.069 | abc-------------- |
| 26 | 18 | 0.410 | 0.181 | abc-------------- |
| 13 | 18 | 0.483 | 0.084 | abcd------------- |
| 29 | 18 | 0.502 | 0.149 | abcd------------- |
| 4 | 18 | 0.528 | 0.205 | Abcde------------ |
| 5 | 18 | 0.536 | 0.205 | Abcde------------ |
| 7 | 18 | 0.553 | 0.154 | abedef----------- |
| 21 | 18 | 0.577 | 0.206 | * |
| 9 | 18 | 0.635 | 0.268 | abcdefg---------- |
| 15 | 18 | 0.725 | 0.271 | abcdefgh--------- |
| 25 | 18 | 0.790 | 0.229 | -bcdefghi-------- |
| 32 | 18 | 0.793 | 0.242 | -bcdefghi-------- |
| 33 | 18 | 0.807 | 0.225 | --cdefghi-------- |
| 35 | 18 | 0.809 | 0.230 | --cdefghi-------- |
| 18 | 12 | 0.819 | 0.217 | --cdefghi-------- |
| 10 | 18 | 0.855 | 0.135 | ---defghi-------- |
| 12 | 18 | 0.958 | 0.357 | ----efghij------- |
| 16 | 18 | 0.988 | 0.290 | -----fghijk------ |
| 1 | 90 | 1.000 | 0.174 | ------ghijk------ |
| 6 | 18 | 1.075 | 0.404 | -------hijkl----- |
| 2 | 18 | 1.078 | 0.150 | -------hijkl----- |
| 31 | 18 | 1.208 | 0.353 | --------ijklm---- |
| 28 | 18 | 1.317 | 0.312 | ---------jklmn--- |
| 8 | 18 | 1.333 | 0.453 | ---------jklmn--- |
| 22 | 18 | 1.403 | 0.380 | ----------klmno-- |
| 30 | 18 | 1.447 | 0.345 | -----------lmno-- |
| 36 | 18 | 1.451 | 0.368 | -----------lmno-- |
| 39 | 18 | 1.468 | 0.653 | -----------lmno-- |
| 20 | 18 | 1.600 | 0.342 | ------------mnop- |
| 38 | 18 | 1.697 | 0.752 | -------------nop- |
| 40 | 18 | 1.733 | 1.112 | * |
| 14 | 18 | 1.806 | 0.386 | --------------op- |
| 37 | 18 | 1.825 | 0.765 | --------------op- |
| 34 | 18 | 1.997 | 0.352 | ---------------p- |
| 27 | 18 | 3.890 | 0.901 | ----------------q |
| Negative | 90 | 0.000 | 0.005 | |

control
n: number of measurements;
μₙₒᵣ: mean normalized expression level (i.e. fold change compared to H1);
σₙₒᵣ: standard deviation of expression level;
Tukey: result of Tukey's studentized range test, haplotypes with overlapping sets of letters are not statistically different in terms of their mean expression level;
*: non-Gaussian distribution

TABLE 4
Haplotype partitioning of GH1 gene promoter expression data

| Haplotype^{§} | leaf^{&} | nₕₐₚ | n | μₙₒᵣ | σₙₒᵣ | δ(leaf) |
|---|---|---|---|---|---|---|
| nnCnnn | 11 | 4 | 72 | 1.809 | 0.725 | 36.27 |
| nGTTnn | 8 | 2 | 108 | 1.067 | 0.267 | 7.62 |
| nTTTGn | 9 | 1 | 18 | 0.635 | 0.268 | 1.22 |
| nTTTAn | 10 | 1 | 18 | 3.890 | 0.902 | 13.82 |
| AnTGnA | 1 | 2 | 36 | 0.418 | 0.142 | 0.71 |
| GnTGnG | 6 | 2 | 36 | 0.607 | 0.262 | 2.39 |
| AnTGnG | 7 | 1 | 18 | 1.825 | 0.765 | 9.95 |
| GTTGGA | 2 | 10 | 174 | 0.740 | 0.427 | 31.54 |
| GGTGAA | 4 | 8 | 144 | 0.735 | 0.474 | 32.16 |
| GGTGGA | 3 | 5 | 90 | 1.035 | 0.493 | 21.66 |
| GTTGAA | 5 | 4 | 72 | 1.178 | 0.384 | 10.47 |

nₕₐₚ: number of haplotypes included in leaf;
μₙₒᵣ: mean normalized expression level;
σₙₒᵣ: standard deviation of expression level;
δ(leaf): residual deviance within leaf;
^{§}alleles are given in the order of SNP 1, 6, 7, 9, 11 and 14 (n: any base);
^{&}numbering as in FIG. 4.

TABLE 5
Linkage disequilibrium, ρ, between GH1
proximal promoter SNPs and LCR
haplotypes in 100 male Caucasians

| | SNP | | | | | | | |
|---|---|---|---|---|---|---|---|---|
| SNP | 4 | 6 | 8 | 9 | 10 | 11 | 12^{&} | 16 |
| 6 | 1.000 | | | | | | | |
| 8 | 0.802 | 0.927 | | | | | | |
| 9 | 0.893 | 0.868 | 1.000 | | | | | |
| 10 | 0.731 | 0.632 | 0.687 | 1.000 | | | | |
| 11 | 0.554 | 0.891 | 0.925 | 0.905 | 0.381 | | | |
| 12^{&} | 0.638 | 0.867 | 0.242 | 1.000 | 1.000 | 1.000 | | |
| 16 | 0.567 | 0.111 | 0.251 | 1.000 | 0.415 | 0.044 | 0.025 | |
| LCR^{§} | 4 | 6 | 8 | 9 | 10 | 11 | 12 | 16 |
| A | 0.153 | 0.829 | 1.000 | 0.931 | 0.601 | 0.782 | 0.800 | 0.064 |
| B | 1.000 | 0.952 | 0.922 | 0.958 | 0.531 | 0.873 | 0.831 | 0.643 |
| C | 0.840 | 0.997 | 0.491 | 0.840 | 0.875 | 0.482 | 1.000 | 0.289 |

^{&}a single chromosome out of 200 was found to carry SNP12 allele C; this chromosome was excluded from all LD analyses involving SNP 12;
^{$}for each LCR haplotype, ρ was calculated against the combination of the other two LCR haplotypes, thereby turning the LCR into a biallelic system.

TABLE 6
Results of EMSA assays that demonstrated
allele-specific differential protein binding
at the various SNP sites in the GH1 gene
promoter using rat pituitary cell nuclear extracts.

| | Position of | | No. of protein interacting | | | Transcription |
|---|---|---|---|---|---|---|
| | double-stranded | Sequence | bands | | | factor binding site/ |
| SNP | oligonucleotide | variation | Strong | Medium | Weak | functional region |
| 8 | −89 → −61 | −75 A | — | 1 | — | Pit-1 |
| | | −75 G | 1 | 1 | — | Pit-1 |
| 9 | −72 → −42 | −57 T | 1 | — | — | Vitamin D receptor |
| | | −57 G | 2 | — | — | Vitamin D receptor |
| 10 | −45 → −15 | −31 G | 1 | — | — | TATA box |
| | | −31 ΔG | — | — | 1 | TATA box |
| 11, 12, 13 | −18 → +15 | −6/−1/+3 | — | — | — | TSS |
| | | AAG | | | | |
| | | −6/−1/+3 | — | — | — | TSS |
| | | GAG | | | | |
| | | −6/−1/+3 | 1 | — | — | TSS |
| | | GTG | | | | |
| 14, 15 | +4 → +37 | +16/+25 | 2 | 1 | — | 5′UTR |
| | | AA | | | | |
| | | +16/+25 | 2 | — | — | 5′UTR |
| | | AC | | | | |
| | | +16/+25 | 1 | — | — | 5′UTR |
| | | GC | | | | |
| | | +16/+25 | 2 | 1 | — | 5′UTR |
| | | GA | | | | |

TSS: Transcriptional start site
5′UTR: 5′ untranslated region

TABLE 7
Association between adult height and GH1 proximal promoter
haplotype-associated in vitro expression data in 124 male
Caucasians

| | Aₓ < 0.9 | Aₓ > 0.9 |
|---|---|---|
| Height < 1.765 | 34 | 22 |
| Height > 1.765 | 21 | 32 |

Aₓ: average normalized in vitro expression level of the two haplotypes of an individual i.e. Aₓ = (μ_{nor, h1} + μ_{nor, h2})/2.

TABLE 8
Average GC cell-derived, normalized luciferase activities ±
standard deviation of different LCR-GH1 proximal promoter
constructs

| Promoter | LCR haplotype | | | |
|---|---|---|---|---|
| haplotype | N | A | B | C |
| H1 | 1.00 ± 0.26^{x} | 2.47 ± 0.41^{yz} | 2.30 ± 0.46^{y} | 2.77 ± 0.55^{z} |
| H23 | 1.00 ± 0.14^{x} | 1.72 ± 0.55^{yz} | 2.14 ± 0.52^{z} | 1.35 ± 0.48^{xy} |
| H27 | 1.00 ± 0.26^{x} | 1.11 ± 0.36^{x} | 1.00 ± 0.41^{x} | 1.25 ± 0.27^{x} |

x, y, z: Tukey's studentized range test within a promoter haplotype; LCR haplotypes (A, B and C) with overlapping sets of letters are not statistically different in terms of their mean expression level. N: Construct containing proximal promoter but lacking LCR. LCR haplotypes were normalised with respect to N in each case.

TABLE 9
Two-way ANOVA of normalized luciferase activities of LCR-GH1
proximal promoter constructs

| Source | df | Mean Square | F Value | p |
|---|---|---|---|---|
| Promoter haplotype | 2 | 51.46 | 390.97 | <0.0001 |
| LCR haplotype | 3 | 5.67 | 43.08 | <0.0001 |
| Interaction | 6 | 3.09 | 23.48 | <0.0001 |

df: degrees of freedom

Supplementary Material

Double-stranded oligonucleotide primer sequences for EMSA analysis of SNP sites exhibiting allele-specific protein binding. SNP sites 11-15 were studied in different allele combinations. TSS: transcriptional initiation site.

| SNP/ | Position | |
|---|---|---|
| allele | from TSS | Sequence 5′→3′ |
| 8 A | −89→−61 | CCATGCATAAATGTACACAGAAACAGGTG |
| | | CACCTGTTTCTGTGTACATTTATGCATGG |
| 8 G | | CCATGCATAAATGTGCACAGAAACAGGTG |
| | | CACCTGTTTCTGTGCACATTTATGCATGG |
| 9 G | −72→−42 | CAGAAACAGGTGGGGGCAACAGTGGGAGAGA |
| | | TCTCTCCCACTGTTGCCCCCACCTGTTTCTG |
| 9 T | | CAGAAACAGGTGGGGTCAACAGTGGGAGAGA |
| | | TCTCTCCCACTGTTGACCCCACCTGTTTCTG |
| 10 G | −45→−15 | GAGAAGGGGCCAGGGTATAAAAAGGGCCCAC |
| | | GTGGGCCCTTTTTATACCCTGGCCCCTTCTC |
| 10 ΔG | | GAGAAGGGGCCAGGTATAAAAAGGGCCCAC |
| | | GTGGGCCCTTTTTATACCTGGCCCCTTCTC |
| 11, 12, 13 | −18→+15 | CCACAAGAGACCAGCTCAAGGATCCCAAGGCCC |
| A A G | | GGGCCTTGGGATCCTTGAGCTGGTCTCTTGTGG |
| 11, 12, 13 | | CCACAAGAGACCGGCTCAAGGATCCCAAGGCCC |
| G A G | | GGGCCTTGGGATCCTTGAGCCGGTCTCTTGTGG |
| 11, 12, 13 | | CCACAAGAGACCGGCTCTAGGATCCCAAGGCCC |
| G T G | | GGGCCTTGGGATCCTAGAGCCGGTCTCTTGTGG |
| 14, 15 | +4→+37 | ATCCCAAGGCCCAACTCCCCGAACCACTCAGGGT |
| A A | | ACCCTGAGTGGTTCGGGGAGTTGGGCCTTGGGAT |
| 14, 15 | | ATCCCAAGGCCCGACTCCCCGCACCACTCAGGGT |
| G C | | ACCCTGAGTGGTGCGGGGAGTCGGGCCTTGGGAT |
| 14, 15 | | ATCCCAAGGCCCGACTCCCCGAACCACTCAGGGT |
| G A | | ACCCTGAGTGGTTCGGGGAGTCGGGCCTTGGGAT |
| 14, 15 | | ATCCCAAGGCCCAACTCCCCGCACCACTCAGGGT |
| A C | | ACCCTGAGTGGTGCGGGGAGTTGGGCCTTGGGAT |

We claim:

1. A method for diagnosing the existence of, or a susceptibility to, growth hormone dysfunction in an individual comprising:
(a) obtaining a test sample of a nucleic acid molecule encoding the proximal promoter region of the growth hormone gene (GH1) from an individual to be tested;
(b) examining said nucleic acid molecule for a plurality of the following six SNP's: 1, 6, 7, 9, 11 and 14 (described in Table 1), or the corresponding haplotypes thereof (also described in Table 1); or a polymorphism in linkage disequilibrium therewith;
(c) and where a plurality of said SNP's, or their said corresponding haplotypes, or their said corresponding polymorphisms, exist determining that the individual may be suffering from, or has a susceptibility to, growth hormone dysfunction.

2. A method according to claim 1 wherein said polymorphism is at 114 of the locus control region of the said gene.

3. A method according to claim 1 wherein said polymorphism is at 1194 of the locus control region of said gene.

4. A method for diagnosing the existence of, or a susceptibility to, growth hormone dysfunction in an individual, comprising:
(a) obtaining a test sample of a nucleic acid molecule encoding the proximal promoter region of the growth hormone gene (GH1) from an individual to be tested;
(b) examining said nucleic acid molecule for any one or more of the following haplotypes in Table 1 indicated as numbers 3, 4, 5, 7, 11, 13, 17, 19, 23, 24, 26 or 29;
(c) and where said haplotypes exist determine that the individual may be suffering from, or has a susceptibility to, growth hormone dysfunction.

5. A method according to claim 1 wherein said examining step under (b) above comprises PCR amplification of said gene.

6. A method according to claim 5 wherein the primer is selected from the group consisting of:
GGG AGC CCC AGC AAT GC (GH1F);
and
TGT AGG AAG TCT GGG GTG C (GH1R).

7. A method according to claim 6 wherein said primers are labelled in order to facilitate detection of the amplified product.

8. A kit suitable for carrying out diagnostic methods of claim 1, which kit comprises:
(a) at least one of the following primers for detecting and/or amplifying the proximal promoter region of the growth hormone gene (GH1);
GGG AGC CCC AGC AAT GC (GH1F);
TGT AGG AAG TCT GGG GTG C (GH1R);
and, optionally,
(b) one or more reagents suitable for carrying out PCR for amplifying desired regions of the patient's DNA.

9. A kit according to claim 8 wherein additional primers are used that are complementary to selected regions of the gene containing the SNP's defined herein as 1, 6, 7, 9, 11 and 14.

10. A vector comprising at least the proximal promoter region of GH1 wherein said region comprises a plurality of the following SNP's: 1, 6, 7, 9, 11 and 14.

11. A vector according to claim 10 wherein said region comprises SNP's 6 and 9.

12. A vector comprising at least the proximal promoter region of GH1 wherein said region comprises SNP's 10 and 12.

13. A vector comprising at least the proximal promoter region of GH1 wherein said region comprises SNP's 8 and 11.

14. A vector according to claim 10 wherein said region is characterised by any one or more of the following haplotypes shown in Table 1: 3, 4, 5, 7, 11, 13, 17, 19, 23, 24, 26 or 29.

15. A vector according to claim 10 which further comprises a GH1 locus control region proximal promoter fusion construct.

16. A vector according to claim 10 wherein said proximal promoter region is functionally linked to the coding region of a selected gene wherein the activity of the said proximal promoter can be monitored.

17. A vector according to claim 16 wherein said proximal promoter region is linked to the coding region of the growth hormone gene (GH1).

18. A vector according to claim 16 wherein said proximal promoter region in said gene is further linked to a tag whereby the expression of said gene, and so the activity of said proximal promoter region, can be monitored.

19. A vector according to claim 18 wherein said tag is a protein tag.

20. A vector according to claim 10 which is further provided with at least one further proximal promoter region of the growth hormone gene (GH1).

21. A vector according to claim 20 wherein said additional proximal promoter region differs from that of the original proximal promoter region.

22. A vector according to claim 21 wherein each proximal promoter region is linked to a different coding sequence.

23. A vector according to claim 21 wherein each proximal promoter region is linked, either directly or indirectly, to a different tag that is capable of monitoring the activities of each of the said promoters.

24. A host cell transformed with a vector according to claim 10.

25. A host cell transformed with a vector according to claim 15.

26. A host cell transformed with a vector according to claim 16.

27. A host cell transformed with a vector according to claim 20.

28. A recombinant cell line that is engineered to express a reporter molecule whose expression is under the control of the proximal promoter of the growth hormone gene wherein said proximal promoter comprises a plurality of the following SNP's: 1, 6, 7, 9, 11 or 14 and/or any one or more of the following haplotypes: 3, 4, 5, 7, 11, 13, 17, 19, 23, 24, 26 or 29 shown in Table 1.

29. A transgenic non-human animal which under expresses growth hormone as a result of having a GH1 promoter containing a plurality of the following SNP's: 1, 6, 7, 9, 11 and 14 and/or as a result of said promoter being characterised by one of the following haplotypes: 3, 4, 5, 7, 11, 13, 17, 19, 23, 24, 26 or 29, shown in Table 1.

30. A transgenic non-human animal according to claim 29 wherein said promoter is characterised by haplotype 23.

31. A transgenic non-human animal wherein the animal expresses growth hormone as a result of having a GH1 promoter characterised by haplotype 27.

32. A transgenic non-human animal wherein the animal expresses growth hormone as a result of having a GH1 promoter characterised by haplotype 1.

33. An artificial proximal promoter region of the growth hormone gene (GH1) characterised by the haplotype AGGGGTTAT-ATGGAG.

34. An artificial proximal promoter region of the growth hormone gene (GH1) characterised by the haplotype AG-TTGTGGGACCACT.

35. An artificial proximal promoter region of the growth hormone gene (GH1) characterised by the haplotype AG-TTTTGGGGCCACT.

36. A method for screening therapeutically active drugs which can be used to treat growth hormone dysfunction comprising exposing a cell or cell line according to claim 24, to a candidate drug and then determining if the candidate drug has affected the activity of the promoter region of the growth hormone gene and so, in the case of the cell line, the expression of the reporter molecule.

37. A method for screening therapeutically active drugs which can be used to treat growth hormone dysfunction comprising exposing a cell or cell line according to claim 28, to a candidate drug and then determining if the candidate drug has affected the activity of the promoter region of the growth hormone gene and so, in the case of the cell line, the expression of the reporter molecule.

38. A method for screening for therapeutically active drugs which can be used to treat growth hormone dysfunction comprising exposing a transgenic non-human animal of claim 29, to candidate drugs and then monitoring the growth of said animal and where the candidate drug is shown to have a positive effect, in terms of animal growth, concluding that said growth is indicative of the therapeutic activity of said candidate drug.


SEQUENCE LISTING (40 sequences)

SEQ ID NO: 1 - DNA, 16 bp, human
ggggggtatgaagaat

SEQ ID NO: 2 - DNA, 16 bp, human
gggggttagggagaat

SEQ ID NO: 3 - DNA, 16 bp, human
gggttgtaggaagaat

SEQ ID NO: 4 - DNA, 16 bp, human
  misc_feature (10)..(10): note = n may be any nucleotide
gggttgtagnaagaat

SEQ ID NO: 5 - DNA, 16 bp, human
gggggttggggagaat

SEQ ID NO: 6 - DNA, 16 bp, human
  misc_feature (10)..(10): note = n may be any nucleotide
gggttgtagnaagaag

SEQ ID NO: 7 - DNA, 16 bp, human
gggggttagggtgaat

SEQ ID NO: 8 - DNA, 16 bp, human
gggttgtagggagaat

SEQ ID NO: 9 - DNA, 16 bp, human
gggggttatggagaat

SEQ ID NO: 10 - DNA, 16 bp, human
  misc_feature (10)..(10): note = n may be any nucleotide
gggttgtagngagaat

SEQ ID NO: 11 - DNA, 16 bp, human
gggggttggggaggct

SEQ ID NO: 12 - DNA, 16 bp, human
gggggttaggaagaat

SEQ ID NO: 13 - DNA, 16 bp, human
  misc_feature (3)..(3): note = n may be any nucleotide
ggnggttggggagaat

SEQ ID NO: 14 - DNA, 16 bp, human
gggggtcagggtgaat

SEQ ID NO: 15 - DNA, 16 bp, human
gggttgtagggtgaat

SEQ ID NO: 16 - DNA, 16 bp, human
gggggttgggaagaat

SEQ ID NO: 17 - DNA, 16 bp, human
  misc_feature (3)..(3): note = n may be any nucleotide
ggnggttagggagaat

SEQ ID NO: 18 - DNA, 16 bp, human
  misc_feature (10)..(10): note = n may be any nucleotide
gggggttagngagaat

SEQ ID NO: 19 - DNA, 16 bp, human
aggggttagggagaat

SEQ ID NO: 20 - DNA, 16 bp, human
  misc_feature (10)..(10): note = n may be any nucleotide
ggggggtagnaagaat

SEQ ID NO: 21 - DNA, 16 bp, human
gggggttggggagaag

SEQ ID NO: 22 - DNA, 16 bp, human
gggttgtatgaagaat

SEQ ID NO: 23 - DNA, 16 bp, human
ggggggtaggaagaat

SEQ ID NO: 24 - DNA, 16 bp, human
  misc_feature (10)..(10): note = n may be any nucleotide
gggttgtggnaagaat

SEQ ID NO: 25 - DNA, 16 bp, human
gggttgtaggaagaag

SEQ ID NO: 26 - DNA, 16 bp, human
gggggttggggtgaat

SEQ ID NO: 27 - DNA, 16 bp, human
gggggttatgaagaat

SEQ ID NO: 28 - DNA, 16 bp, human
  misc_feature (10)..(10): note = n may be any nucleotide
gggggttagnaagaat

SEQ ID NO: 29 - DNA, 16 bp, human
aggggttaggaagaat

SEQ ID NO: 30 - DNA, 16 bp, human
  misc_feature (3)..(3): note = n may be any nucleotide
ggnggttaggaagaat

SEQ ID NO: 31 - DNA, 16 bp, human
  misc_feature (10)..(10): note = n may be any nucleotide
gggggttggngagaat

SEQ ID NO: 32 - DNA, 16 bp, human
gggttgtggggagaag

SEQ ID NO: 33 - DNA, 16 bp, human
gggggttagggaggct

SEQ ID NO: 34 - DNA, 16 bp, human
  misc_feature (3)..(3): note = n may be any nucleotide
ggnggtcagggtgaat

SEQ ID NO: 35 - DNA, 16 bp, human
ggggggtaggaccaat

SEQ ID NO: 36 - DNA, 16 bp, human
gggggttagggtgaag

SEQ ID NO: 37 - DNA, 16 bp, human
aggggttagggaggat

SEQ ID NO: 38 - DNA, 16 bp, human
gggggtcaggaagaat

SEQ ID NO: 39 - DNA, 16 bp, human
gggttgtagggagact

SEQ ID NO: 40 - DNA, 16 bp, human
gggggtcagggagaat